(19) United States
(12) Patent Application Publication
(10) Pub. No.: US 2004/0265877 A1
(43) Pub. Date: Dec. 30, 2004

(54) Methods for detecting presence of cellular constituents

(75) Inventors: Marvin S. Antelman, Rehovot (IL); Perry W. Antelman, Sharon, MA (US)

Correspondence Address: BANNER & WITCOFF, LTD., 28 STATE STREET, 28th FLOOR, BOSTON, MA, 02109-9601, US

(73) Assignee: Marantech Holding, LLC, Providence, RI

(21) Appl. No.: 10/833,490

(22) Filed: Apr. 28, 2004

Related U.S. Application Data
(63) Continuation-in-part of application No. PCT/US03/13033, filed on Apr. 28, 2003.

(51) Int. Cl. C12Q 1/68; G01N 33/53; G01N 33/567; G01N 33/554; G01N 33/569

(52) U.S. Cl. 435/6; 435/7.2; 435/7.32

(57) ABSTRACT

The invention is directed to methods for detecting, monitoring and/or diagnosing aberrant cellular proliferation, and disorders associated therewith, such as cancer, and/or infection by microorganisms. The detection, monitoring and/or diagnosis comprises contacting a compound of the invention with a cell or fluid sample. Compound binding confirms the presence of an abnormal cell or a protein associated with an abnormal cell or infection by one or more microorganisms, and/or disorders associated with infection by one or more microorganisms.

### RELATED APPLICATIONS

This application claims priority to PCT/US03/13033 designating the United States, filed on Apr. 28, 2003, hereby incorporated by reference in its entirety for all purposes.

### BACKGROUND OF THE INVENTION

1. Field of the Invention

The invention relates to methods for detecting the presence of cellular constituents associated with aberrant cellular proliferation, infection by microorganisms and/or disorders associated with aberrant cellular proliferation and/or infection by microorganisms. The compounds used in the present methods are multivalent metal oxides that can bind to the cellular constituents and thereby serve to detect the cellular constituents. Detecting of certain cellular constituents is useful in and of itself as a positive/negative assay, and is also useful in the diagnosis of certain diseases, such as cancer, or certain infections by microorganisms.

2. Description of Related Art

Multivalent metal oxides, such as electron active metal oxides including multivalent silver cations, have many disclosed (M. Antelman, “Anti-Pathogenic Multivalent Silver Molecular Semiconductors,” Precious Metals, vol. 16:141-149 (1992); M. Antelman, “Multivalent Silver Bactericides,” Precious Metals, vol. 16:151-163 (1992)). For example, tetrasilver tetroxide activated with an oxidizing agent is disclosed for use in bactericidal, fungicidal, and algicidal use, such as in municipal and industrial water treatment applications and for the treatment of HIV infection.

Certain divalent silver compounds are also disclosed for water treatment typically in combination with certain oxidizing agents, metals, or other compounds, as disinfectants, bactericides, algicides, and fungicides. See, for example, M. Antelman, “Silver (II, III) Disinfectants,” Soap/Cosmetics/Chemical Specialties, pp. 52-59 (March, 1994), and U.S. Pat. Nos. 5,017,295; 5,073,382; 5,078,902; 5,089,275; 5,098,582; 5,211,855; 5,223,149; 5,336,416; and 5,772,896. Multivalent silver antimicrobials are also disclosed in U.S. Pat. No. 5,017,295 for Ag(II) and U.S. Pat. No. 5,223,149 for Ag (III).

U.S. Pat. No. 5,336,499 discloses tetrasilver tetroxide and persulfate compositions having certain in vitro anti-pathogenic properties, i.e., bactericidal, fungicidal, viricidal, and algicidal, in certain concentrations as low as 0.3 ppm, particularly in nutrient broth cultures. The persulfate is disclosed to be an oxidizing agent that activates the tetroxide crystals.

In vitro assays, such as those disclosed in Ahmed, S. A., Gogal Jr., R. M. and Walsh, J. E., a New Rapid and Simple Non-radioactive Assay to Monitor and Determine the Proliferation of Lymphocytes: an Alternative to ³H-thymidine Incorporation Assay, Journal of Immunological Methods 1994; 170: 211-224; Boyd, M. R., Status of the NCI Preclinical Antitumor Drug Discovery Screen, J. B. Lippincott Company, Philadelphia, Principles & Practices of Oncology Updates 1989; 3 # 10: 1-12, and Boyd, M. R. et al. Data Display and Analysis Strategies for the NCI Disease-oriented in vitro Antitumor Drug Screen in Cytotoxic Anti-cancer Drugs: Models and Concepts for Drug Discovery and Development, Kluwer Academic, Boston, 1992: 11-34; have been used to estimate the cytotoxicity of anti-cancer therapeutics.

U.S. Pat. No. 5,571,520 discloses the use of molecular crystals of tetrasilver tetroxide, particularly with oxidizing agents to enhance the efficiency of such devices, for killing pathogenic microorganisms, such as staphylococcal infections. Amounts of 10 ppm sodium persulfate as an oxidizing agent were used with certain amounts of silver tetroxide in the reported in vitro testing. One human study involved in vivo curing of a gynecological yeast infection with 10 ppm of the silver tetroxide and 40 ppm sodium persulfate. Other in vivo topical studies report in conclusory fashion the cure of a single case of athlete's foot with a solution of 100 ppm of the composition and the cure of a single case of toenail fungus with a 25% suspension of the composition.

U.S. Pat. No. 5,676,977 discloses intravenously injected tetrasilver tetroxide crystals used for destroying the HIV virus, AIDS synergistic pathogens, and immunity suppressing moieties (ISM) in humans. The crystals were formulated for a single injection at about 40 ppm of human blood. This reference also discloses that the compositions cause hepatomegaly, also known as enlarged liver, albeit with no reported loss of liver function.

The aforementioned references report detailed descriptions of the mechanism via which the multivalent silver molecular crystal devices were believed to operate. A discussion of such results and concepts was also presented at a Seminar entitled “Incurable Diseases Update” (Weizmann Institute of Science, Rehovot, Israel, Feb. 11, 1998, “Beyond Antibiotics, Non Toxic Disinfectants and Tetrasil™).

### SUMMARY OF THE INVENTION

Embodiments of the present inventions are based on the discovery that multivalent metal oxide compounds can be used to bind to certain target proteins produced by cells associated with a certain disease or infectious state. Importantly, the target proteins need not be constrained to the cell, but rather may be separate from the cell, such as when an abnormal cell (for example a cancer cell) creates numerous copies of a target protein that then dissociate from the cell and enter the surrounding media, including fluid and normal cells, adjacent the abnormal or infected cell. A suitable sample sufficient to detect the disease or infectious state would therefore not require presence of the actual abnormal or infected cell, which may be difficult to obtain, but would at a minimum only require presence of the target protein produced by the abnormal or infected cell. Given that numerous copies of the target protein may be spread outside of the single abnormal or infected cell, likelihood of detection of the presence of the abnormal or infected cell is enhanced. This aspect of the present invention creates significant advantages in reducing the number of false negatives in assays requiring an actual abnormal or infected cell in the test sample when the test sample lacks such an actual abnormal or infected cell.

According to the method of the present invention, a tissue or fluid sample is obtained from an animal, including a human, and the tissue or fluid sample is contacted with the multivalent metal oxide compounds of the present invention. Binding is then allowed to occur between the multivalent metal oxide compounds and one or more target proteins. As a result of the binding, which may be covalent, the multivalent metal oxide compound releases one or more electrons creating an electrical charge. This electrical charge can be detected by application of a suitable reagent that produces either a visual color change or another detectable change in the sample. The color change may be detected by visual or spectrophotometric methods known to those skilled in the art. Alternate methods for detecting the binding of the multivalent metal oxides are described below and will become apparent to those of skill in the art.

According to certain embodiments of the present invention, target proteins are specific to certain abnormal or infected cell types. Target proteins include those having moieties such as NH, NH₂, S—S and SH. One particular target protein having an SH group useful in the assays of the present invention is tNOX (Morre (2003) Free Radical Research 37: tNOX is believed to be specific to all forms of cancer cells. The binding of a multivalent metal oxide to tNOX has been described in the literature. One of ordinary skill in the art will be able to identify other target proteins based on the description herein.

The target proteins may be intracellular, located on the cell surface or they may be produced by the cell and then discharged exterior to the cell into the surrounding media. The multivalent metal oxides are attracted to the nitrogen and/or sulfur moieties on the target protein and become covalently attached to the target protein. Without wishing to be bound to any particular scientific theory, it is believed that the nitrogen and/or sulfur moieties act as conduits for the transfer of electrons from the multivalent metal oxides' low valence ions to the high valence ions. According to a specific embodiment, it is believed that sulfhydryl groups act as conduits for the transfer of electrons from tetrasilver tetroxide's monovalent silver ions to the trivalent ions in oxidation-reduction reactions. This action yields divalent silver ions that bind covalently to certain ectoproteins producing an electrically-charged surface enabling for rapid detection using the assays described herein.

Additional embodiments of the present invention are directed to assays for detecting and/or monitoring abnormal cellular proliferation and/or the presence of microorganisms (e.g., viruses, bacteria, fungi, parasites and the like) and/or diagnosing and/or monitoring disorders associated with aberrant cellular proliferation (e.g., cancer) and/or disorders associated the presence of microorganisms. According to this aspect of the present invention, the target protein is associated with a particular disease or infectious state. Detection of the particular target protein by a multivalent metal oxide compound is then used to diagnose or monitor a particular disease or infectious state.

According to another aspect of the present invention, methods are provided for screening candidate compounds for their effect on the abnormal or infected cell to reproduce and/or produce certain target proteins. The candidate compounds may have the ability to kill cells entirely, thereby preventing their replication and/or production of target proteins. Candidate compounds may also effect the ability of the cells to replicate and/or produce the target protein, while maintaining the viability of the cell. For example, candidate compounds may upregulate or downregulate cellular proliferation and/or the ability of the cell to produce the target protein. According to this aspect of the present invention, the level of binding of the multivalent metal oxide compounds to the target proteins is compared between a standard sample of the abnormal or infected cell and a test sample of the abnormal or infected cell that has been contacted with a candidate compound. A greater color change in the test sample indicates the presence of more cells or target proteins compared to the standard. A lesser color change in the test sample indicates the presence of fewer cells or target proteins compared to the standard.

In accordance with an additional embodiment of the present invention, assay kits are provided for the detection of aberrant cellular proliferation and/or infection by a microorganism. In another embodiment, kits are provided for the diagnosis of disorders associated with aberrant cellular proliferation and/or a disorder associated with infection by a microorganism. In one aspect, the kits comprise a multivalent metal oxide, and optionally, instructions for use.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIGS. 1A-1C depict Pap (Papanicolaou test) smear slides stained with tetrasilver tetroxide reagent. A depicts negative control cells; B depicts low-grade squamous intraepithelial lesion (LSIL) cells; C depicts cells of a specimen diagnosed as a high-grade squamous intraepithelial lesion (HSIL) cell lesion. B and C may indicate the presence of human papilloma virus (HPV), which is known to be a precursor of cervical cancer.

FIG. 2 depicts a slide of a stained sample of malignant melanoma showing both malignant tissue (darkly stained region) and non-malignant tissue (unstained region). Magnification: 40×.

FIG. 3A-3D depict slides of skin lesions prior to staining (A and B) and subsequent to staining (C and D). A and C show a non-malignant nevi sample and B and D show a malignant melanoma sample. Dark staining was observed in part D after enhancement.

FIG. 4 depicts Pap smear samples stained with tetrasilver tetroxide reagent. HeLa (cervical carcinoma) cells depict black staining of nuclei. Normal cells depict no nuclear staining. Herpes virus infected cells depict no nuclear staining, but show a brown staining of the infected cells.

FIGS. 5A-5C depict cervical carcinoma cells and breast cancer cells stained with tetrasilver tetroxide reagent and an enhancer. A depicts HeLa cells, B depicts BT-20 mammary cancer cells and C depicts MCF-10A (non-cancer) mammary cells.

FIGS. 6A-6B depict slides of cells stained with tetrasilver tetroxide reagent. A depicts cells that are positive for cancer. B depicts negative control, atypical squamous cells of uncertain significance (ASCUS), LGSIL and HGSIL cells.

FIG. 7 depicts a scanning electron micrograph (SEM) of HeLa cell sections stained with tetrasilver tetroxide reagent for one hour. The micrograph depicts numerous tetrasilver tetroxide particles bound to the cell surface. The micrograph also depicts crystalline staining material bound to the interior portions of the sectioned HeLa cells.

FIGS. 8A-8E depict frozen liver slices stained with tetrasilver tetroxide reagent from a wild type mouse (non-cancerous) (A-C) and a transgenic mouse expressing tNOX on its liver cells (D and E). After computer photography enhancement, the transgenic liver showed black nuclei and black borders whereas the wild type mouse liver did not. 40× magnification.

FIGS. 9A-9C depict SEMs of HeLa cells and MCF-10A (non-cancerous) cells. A depicts a HeLa cell stained with Fe₃O₄. B depicts an unstained HeLa cell. C depicts an MCF-10A cell treated with tetrasilver tetroxide reagent.

FIGS. 10A-10B depict SEMs of MCT-10A cells with (A) or without (B) treatment with tetrasilver tetroxide reagent. The non-cancer cells (A) show no tetrasilver tetroxide binding.

### DETAILED DESCRIPTION OF CERTAIN EXAMPLES

The present invention is directed in part to the discovery that the compounds described herein (e.g., multivalent metal oxides) can be used to detect the presence of aberrant cellular proliferation and/or infection by one or more microorganisms as well as to diagnose disorders associated with aberrant cellular proliferation (e.g., cancer) and/or disorders associated with infection by a microorganism. Embodiments of the present invention are thus directed to methods of detecting the presence of aberrant cellular proliferation and/or one or more microorganisms in a sample as well as methods of diagnosing disorders associated with aberrant cellular proliferation and/or disorders associated with infection by a microorganism.

In one embodiment, compounds of the invention are multivalent metal oxides. Examples of suitable multivalent metal oxides include, but are not limited to: compounds comprising tetrasilver tetroxide (Ag₄O₄), comprised of monovalent silver ions (i.e., Ag(I)) and trivalent silver ions (i.e., Ag(III)) (multiple valence: Ag (I,III); dibismuth tetroxide (Bi₂O₄), comprised of trivalent bismuth ions (i.e., Bi(III)) and pentavalent bismuth ions (i.e., Bi(V)) (multiple valence: Bi (III,V)); tricobalt tetroxide (CO₃O₄), comprised of divalent cobalt ions (i.e., Co(II)) and trivalent cobalt ions (i.e., Co(III)) (multiple valence: Co(II,III)); tetracopper tetroxide (CU40₄), comprised of monovalent copper ions (i.e., Cu(I)) and trivalent copper ions (i.e., (Cu(III)) (multiple valence: Cu(I,III)); triiron tetroxide (Fe₃O₄), comprised of divalent iron ions (i.e., Fe(II)) and trivalent iron ions (i.e., (Fe(III)) (multiple valence: Fe(II,III)); trimanganese tetroxide (Mn₃O₄), comprised of divalent manganese ions (i.e., Mn(II)) and trivalent manganese ions (i.e., Mn(III)) (multiple valence: Mn(II,III)); hexapraseodymium octoxide (Pr₆O₁₁), comprised of trivalent praseodymium ions (i.e., Pr(III)) and tetravalent praseodymium ions (i.e., Pr(IV)) (multiple valence: Pr(III,IV)); and tetraterbium heptoxide (Tb₄O₇), comprised of trivalent terbium ions (i.e., (Tb(III)) and tetravalent terbium ions (i.e., (Tb(IV)) (multiple valence: Tb(III,IV)). One of ordinary skill in the art will be able to identify other suitable multivalent metal oxide compounds useful in the practice of the present invention based on the disclosure herein.

The compounds (e.g., multivalent metal oxides) of the present invention, without intending to be bound by theory, are believed to have unique crystal structures in that, in the case of the metal oxides, there are generally atoms of the same element in the crystal that have at least two different valences as indicated above, typically at least one lower valent metal cation and at least one higher-valent metal cation, for example, such as Co(II) and Co(III), respectively. Exemplary electron active metal oxide compounds according to the invention include, but are not limited to, Ag(I,III), Co(II,III), Pr(III,IV), Bi(III,V), Fe(II,III), Mn(II,III), Cu(I,III) and Tb(III,IV) oxides. Without intending to be bound by theory, it is believed that the electron active compounds interact with pathogens by transferring electrons between their lower valent ions and their higher valent ions in the crystal.

Further, without intending to be bound by theory, it is thought that multivalent metal oxides such as tetrasilver tetroxide are attracted to the sulfhydryl groups of select ectoproteins which are commonly found on the surface membrane of all cancer cells. Sulfhydryl groups act as conduits for the transfer of electrons from tetrasilver tetroxide's monovalent silver ions to the trivalent ions in oxidation-reduction reactions. It is believed that this action yields divalent silver ions that bind covalently to the ectoproteins, producing an electrically-charged surface enabling for rapid detection using the assays described herein. Alternatively, the silver ions that are bound to the target proteins themselves may be detectable by spectrophotometry or other reagents or by other means known to those skilled in the art.

By detection of certain target proteins associated with certain abnormal cells associated with certain disease states, the assays described herein can be used for the detection of aberrant cellular proliferation and/or diagnosis of disorders associated with aberrant cellular proliferation (e.g., cellular proliferative disorders such as cancer). As used herein, the term “cellular proliferative disorder” includes disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition (1995)). The terms “neoplasm” and “tumor” refer to an abnormal tissue that grows by cellular proliferation more rapidly than normal and continues to grow after the stimuli that initiated proliferation is removed (see, for example, PDR Medical Dictionary 1st edition (1995)). Such abnormal tissue shows partial or complete lack of structural organization and functional coordination with the normal tissue which may be either benign (i.e., benign tumor) or malignant (i.e., malignant tumor).

The language “diagnosis of cellular proliferative disorders” is intended to include the identification of the presence and/or growth of neoplasms in a subject or metastasis of a neoplasm from one site to another. Examples of the types of neoplasms intended to be encompassed by the present invention include but are not limited to those neoplasms associated with cancers of the breast, skin, bone, prostate, ovaries, uterus, cervix, liver, lung, brain, larynx, gallbladder, pancreas, rectum, parathyroid, thyroid, adrenal gland, immune system, neural tissue, head and neck, colon, stomach, bronchi, and/or kidneys.

In accordance with certain other examples, the assays described herein can be used for the detection of infection and/or the diagnosis of a disorder associated with infection of a cell, tissue, organ and the like by one or more microorganism including, but not limited to, viruses, bacteria, fungi, parasites and the like. Viruses include, but are not limited to, DNA or RNA animal viruses. As used herein, RNA viruses include, but are not limited to, virus families such as picomaviridae (e.g., polioviruses), reoviridae (e.g., rotaviruses), togaviridae (e.g., encephalitis viruses, yellow fever virus, rubella virus), orthomyxoviridae (e.g., influenza viruses), paramyxoviridae (e.g., respiratory syncytial virus, measles virus, mumps virus, parainfluenza virus), rhabdoviridae (e.g., rabies virus), coronaviridae, bunyaviridae, flaviviridae, filoviridae, arenaviridae, bunyaviridae, and retroviridae (e.g., human T-cell lymphotropic viruses (HTLV), human immunodeficiency viruses (HIV)). As used herein, DNA viruses include, but are not limited to, virus families such as papovaviridae (e.g., papilloma viruses), adenoviridae (e.g., adenovirus), herpesviridae (e.g., herpes simplex viruses), and poxyiridae (e.g., variola viruses).

Bacteria include, but are not limited to, gram positive bacteria, gram negative bacteria, acid-fast bacteria and the like. As used herein, gram positive bacteria include, but are not limited to, Actinomedurae, Actinomyces israelii, Bacillus anthracis, Bacillus cereus, Clostridium botulinum, Clostridium difficile, Clostridium perfringens, Clostridium tetani, Corynebacterium, Enterococcus faecalis, Listeria monocytogenes, Nocardia, Propionibacterium acnes, Staphylococcus aureus, Staphylococcus epiderm, Streptococcus mutans, Streptococcus pneumoniae and the like.

As used herein, gram negative bacteria include, but are not limited to, Afipia felis, Bacteriodes, Bartonella bacilliformis, Bortadella pertussis, Borrelia burgdorferi, Borrelia recurrentis, Brucella, Calymmatobacterium granulomatis, Campylobacter, Escherichia coli, Francisella tularensis, Gardnerella vaginalis, Haemophilus aegyptius, Haemophilus ducreyi, Haemophilus influenziae, Heliobacter pylori, Legionella pneumophila, Leptospira interrogans, Neisseria meningitidia, Porphyromonas gingivalis, Providencia sturti, Pseudomonas aeruginosa, Salmonella enteridis, Salmonella typhi, Serratia marcescens, Shigella boydii, Streptobacillus moniliformis, Streptococcus pyogenes, Treponema pallidum, Vibrio cholerae, Yersinia enterocolitica, Yersinia pestis and the like.

As used herein, acid-fast bacteria include, but are not limited to, Myobacterium avium, Myobacterium leprae, Myobacterium tuberculosis and the like.

As used herein, other bacteria not falling into the other three categories include, but are not limited to, Bartonella henseiae, Chlamydia psittaci, Chlamydia trachomatis, Coxiella burnetii, Mycoplasma pneumoniae, Rickettsia akari, Rickettsia prowazekii, Rickettsia rickettsii, Rickettsia tsutsugamushi, Rickettsia typhi, Ureaplasma urealyticum, Diplococcus pneumoniae, Ehrlichia chafensis, Enterococcus faecium, Meningococci and the like.

As used herein, fungi include, but are not limited to, Aspergilli, Candidae, Candida albicans, Coccidioides immitis, Cryptococci and combinations thereof.

As used herein, parasitic microbes include, but are not limited to, Balantidium coli, Cryptosporidium parvum, Cyclospora cayatanensis, Encephalitozoa, Entamoeba histolytica, Enterocytozoon bieneusi, Giardia lamblia, Leishmaniae, Plasmodii, Toxoplasma gondii, Trypanosomae, trapezoidal amoeba, Trichomonas vaginalis and the like.

In accordance with certain examples, the compounds described herein can be used for the detection of a target protein associated with aberrant cellular proliferation and/or infection by a microorganism, as well as the diagnosis of disorders associated with aberrant cellular proliferation and/or disorders associated with infection by a microorganism, by contacting the compound with a sample and assaying binding of the compound to one or more target proteins in the sample. As used herein, the terms “bind,” “binding,” “interact” and “interacting” refer to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions are much weaker than covalent interactions, but play a major role in determining the three-dimensional structure of macromolecular structures. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994.

The metal ion of certain electron active compounds can exhibit a distinct affinity for certain elements of ligands, for example, such as sulfur, oxygen, or nitrogen, particularly when the ligands are present in or on a cell membrane. Without intending to be bound by theory, in many cases, the metal ion will not merely bind to these elements, but will actually form chelate complexes with their ligands. The classic example of this is Ag(I,III) oxide, the monovalent silver ion of which has an affinity for sulfur and nitrogen and the oxidized/reduced divalent ion of which forms chelate complexes with, for example, mercapto or amino groups. Thus, the electron active compound attraction for the cell membrane surfaces, for example, of pathogens, is, without intending to be bound by theory, believed to be driven by powerful electrostatic forces.

Multivalent Metal Oxides

Multivalent metal oxide compounds useful in the present invention include, but are not limited to, those described in detail in U.S. Pat. No. 6,645,531, hereby incorporated by reference in its entirety for all purposes. Most of the metal oxide compounds for use according to the invention are commercially available from various sources. Tetrasilver tetroxide compositions for use according to the invention are commercially available from Aldrich Chemical Co., Inc. (Milwaukee, Wis.). Alternatively, the chemical synthesis of tetrasilver tetroxide compounds is described in the art. See U.S. Pat. No. 5,336,499, incorporated herein by reference in its entirety for all purposes.

Fe(II,III) oxide and Mn(II,II) oxide are commercially available from Aldrich Company (Milwaukee, Wis.), and Co(II,III) oxide and Pr(III,IV) oxide are commercially available from Noah Technologies (San Antonio, Tex.). Bi(III,V) oxide synthetic routes are detailed and reviewed in Gmelins Handbuch Der Anorganischen Chemie, vol. 16:642 (1964) (incorporated herein by reference in its entirety for all purposes), and the oxide is available commercially from City Chemicals of New York, N.Y.

Methods of making multivalent metal oxides are further described in U.S. Pat. No. 6,645,531, hereby incorporated herein by reference in its entirety for all purposes.

The multivalent metal oxide compounds useful in the present invention can be formulated into aqueous solutions or other liquid vehicles, pharmaceutical or otherwise, known to those of skill in the art.

Diagnostic Assays

An exemplary method for detecting and/or diagnosing aberrant cellular proliferation (e.g., cancer) and/or the presence of one or more microorganisms in a biological sample involves obtaining a biological sample from a test subject and contacting the biological sample with one or more of the compounds described herein (e.g., multivalent metal oxides) using the assays described below. Compounds binding to a target protein present in the sample can then be detected to confirm presence of the target protein in the sample and, accordingly, presence of the cell in the sample or in the test subject from which the sample was taken.

As used herein, the term “biological sample” is intended to include tissues, cells and biological fluids isolated from a subject, as well as tissues, cells and fluids present within a subject. Biological samples may be of any biological tissue or fluid or cells. Typical biological samples include, but are not limited to, sputum, lymph, blood, blood cells (e.g., white cells), fat cells, cervical cells, cheek cells, throat cells, mammary cells, muscle cells, skin cells, liver cells, spinal cells, bone marrow cells, tissue (e.g., muscle tissue, cervical tissue, skin tissue, spinal tissue, liver tissue and the like) fine needle biopsy samples, urine, cerebrospinal fluid, peritoneal fluid and pleural fluid, or cells therefrom. Biological samples may also include sections of tissues such as frozen sections taken for histological purposes. A biological sample may be obtained from a mammal, including, but not limited to horses, cows, sheep, pigs, goats, rabbits, guinea pigs, rats, mice, gerbils, non-human primates and humans. Biological samples may also include cells from microorganisms (e.g., bacterial cells, viral cells, yeast cells and the like) and portions thereof. As used herein, the term “biological fluid” is intended to include any fluid taken from a biological organism. Biological fluids include, but are not limited to, sputum, lymph, blood, urine, tears, breast milk, nipple aspirate fluid, seminal fluid, vaginal secretions, feces (e.g., runny stool), cerebrospinal fluid, peritoneal fluid, pleural fluid, pus, ascites and the like.

In another embodiment, the methods of the invention further involve obtaining a control biological sample from a control subject, contacting the control sample with one or more of the compounds described herein, and comparing the reactivity (e.g., binding) of the compound to the control sample with the reactivity of the compound (e.g., binding) to a test sample.

In another embodiment, the level of binding of the compound is used quantify the level of aberrant cellular proliferation, or the level of microorganisms present, as well as to follow the progression and/or regression of a disorder associated with aberrant cell proliferation and/or infection with a microorganism. In one aspect, the invention provides a spectrum (e.g., a spectrum of color (e.g., shading), vibrational energy and the like), wherein the amount of compound bound may be quantified by comparison with the spectrum. For example, a spectrum may range in color from white to tan to brown to black, wherein white corresponds to no or very little compound bound, tan and brown correspond to quantified intermediate levels of compound bound and black corresponds to a maximal level of compound bound. Various other colors (e.g., red, yellow, orange and the like) could be detected as well. These spectra may be further refined using the enhancement methods described herein.

In another embodiment, the methods of the invention further relate to use of the compounds described herein (e.g., multivalent metal oxides) in vivo (e.g., in or on an organism such as a mammal, e.g., a human) using pharmaceutically acceptable compositions. Pharmaceutical compositions and methods of administering pharmaceutical compositions are described further herein. Binding of the compound in vivo can be assayed visually, or using imaging techniques known in the art such as x-rays, computed tomography (CT) scans, magnetic resonance imaging (MRI), positron emission tomography (PET) scans, single photon emission computed tomography (SPECT), magnetic resonance spectroscopy imaging (MRSI) and the like. Suitable imaging techniques would be readily understood by one of skill in the art.

The invention also encompasses kits for detecting the presence of aberrant cellular proliferation and/or the presence of one or more pathogenic organisms in a biological sample as well as for diagnosing disorders associated with aberrant cellular proliferation and/or the presence of one or more pathogenic organisms. For example, the kit can comprise one or more of the compounds described herein capable of detecting aberrant cellular proliferation or the presence of microorganisms in a biological sample; means for determining the binding of the compound to the sample; and means for comparing the binding of the sample with a standard.

The compound can be packaged in a suitable container. The kit can further comprise instructions for using the kit to detect cancer and/or the presence of a microorganism. Kits according to the present invention include, but are not limited to, at home diagnostic kits as well as diagnostic kits for use in a clinical environment (e.g., hospital, clinic, physician's office and the like), in pathology laboratories, in scientific research laboratories and the like.

Prognostic Assays

The diagnostic methods described herein can be utilized to identify subjects having or at risk of developing a disease or disorder associated with aberrant cell proliferation (e.g., cancer) and/or infection with a microorganism. Thus, the present invention provides a method for identifying a disease or disorder associated with aberrant cell proliferation and/or infection with one or more microorganisms in which a test sample is obtained from a subject and compound binding is detected, wherein the presence or increased levels of compound binding is diagnostic for a subject having or at risk of developing a disease or disorder associated with aberrant cell proliferation and/or infection with one or more microorganisms. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant cell proliferation and/or infection with one or more microorganisms. For example, such methods can be used to determine whether a particular agent inhibits the proliferation of an abnormal or infected cell by comparing the level of binding of the compounds of the present invention in a standard sample versus a test sample which has been contacted with a candidate agent.

Screening Assays

The present invention provides a method (also referred to herein as a “screening assay”) for identifying modulators, i.e., candidate or test compounds or agents (e.g., peptides, cyclic peptides, peptidomimetics, small molecules, small organic molecules, or other drugs) which have a stimulatory or inhibitory effect on cell proliferation and/or infection with one or more microorganisms and/or production of target proteins.

As used herein, the term “small organic molecule” refers to an organic molecule, either naturally occurring or synthetic, that has a molecular weight of more than about 25 daltons and less than about 3000 daltons, preferably less than about 2500 daltons, more preferably less than about 2000 daltons, preferably between about 100 to about 1000 daltons, more preferably between about 200 to about 500 daltons.

The modulators of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412), or on beads (Lam (1991) Nature 354:82), chips (Fodor (1993) Nature 364:555), bacteria (Ladner U.S. Pat. No. 5,223,409), spores (Ladner U.S. Pat. No. 5,223,409), plasmids (Cull et al. (1992) Proc. Natl. Acad. Sci. USA 89:1865) or on phage (Scott and Smith (1990) Science 249:386); (Devlin (1990) Science 249:404); (Cwirla et al. (1990) Proc. Natl. Acad. Sci. USA 87:6378); (Felici (1991) J. Mol. Biol. 222:301); (Ladner supra).

Examples of methods for introducing a molecular library of randomized nucleic acids into a population of cells can be found in the art, for example in U.S. Pat. No. 6,365,344, incorporated herein in its entirety by reference. A molecular library of randomized nucleic acids can provide for the direct selection of candidate or test compounds with desired phenotypic effects. The general method can involve, for instance, expressing a molecular library of randomized nucleic acids in a plurality of cells, each of the nucleic acids comprising a different nucleotide sequence, screening for a cell of exhibiting a changed physiology in response to the presence in the cell of a candidate or test compound, and detecting and isolating the cell and/or candidate or test compound.

In one embodiment, the introduced nucleic acids are randomized and expressed in the cells as a library of isolated randomized expression products, which may be nucleic acids, such as mRNA, antisense RNA, siRNA, ribozyme components, etc., or peptides (e.g., cyclic peptides). The library should provide a sufficiently structurally diverse population of randomized expression products to effect a probabilistically sufficient range of cellular responses to provide one or more cells exhibiting a desired response. Generally at least 10⁶, at least 10⁷, at least 10⁸, or at least 10⁹ different expression products are simultaneously analyzed in the subject methods. In one aspect methods maximize library size and diversity.

The introduced nucleic acids and resultant expression products are randomized, meaning that each nucleic acid and peptide consists of essentially random nucleotides and amino acids, respectively. The library may be fully random or biased, e.g., in nucleotide/residue frequency generally or per position. In other embodiments, the nucleotides or residues are randomized within a defined class, e.g. of hydrophobic amino acids, of purines, etc.

Functional and structural isolation of the randomized expression products may be accomplished by providing free (not covalently coupled) expression product, though in some situations, the expression product may be coupled to a functional group or fusion partner, preferably a heterologous (to the host cell) or synthetic (not native to any cell) functional group or fusion partner. Exemplary groups or partners include, but are not limited to, signal sequences capable of constitutively localizing the expression product to a predetermined subcellular locale such as the Golgi, endoplasmic reticulum, nucleoli, nucleus, nuclear membrane, mitochondria, chloroplast, secretory vesicles, lysosome, and the like; binding sequences capable of binding the expression product to a predetermined protein while retaining bioactivity of the expression product; sequences signaling selective degradation, of itself or co-bound proteins; and secretory and membrane-anchoring signals.

It may also be desirable to provide a partner which conformationally restricts the randomized expression product to more specifically define the number of structural conformations available to the cell. For example, such a partner may be a synthetic presentation structure: an artificial polypeptide capable of intracellularly presenting a randomized peptide as a conformation-restricted domain. Generally such presentation structures comprise a first portion joined to the N-terminal end of the randomized peptide, and a second portion joined to the C-terminal end of the peptide. Preferred presentation structures maximize accessibility to the peptide by presenting it on an exterior loop, for example of coiled-coils, (Myszka and Chaiken (1994) Biochemistry 33:2362). To increase the functional isolation of the randomized expression product, the presentation structures are selected or designed to have minimal biologically active as expressed in the target cell. In addition, the presentation structures may be modified, randomized, and/or matured to alter the presentation orientation of the randomized expression product. For example, determinants at the base of the loop may be modified to slightly modify the internal loop peptide tertiary structure, while maintaining the absolute amino acid identity. Other presentation structures include zinc-finger domains, loops on beta-sheet turns and coiled-coil stem structures in which non-critical residues are randomized; loop structures held together by cysteine bridges, cyclic peptides, etc.

In another embodiment, the present invention provides cyclic peptides for use in the libraries described herein. As used herein, the term “cyclic peptide” refers to a peptide configured in a loop. Cyclic peptides can be produced by generating a nucleotide sequence encoding a peptide to be cyclized flanked on one end with a nucleotide sequence encoding the carboxy-terminal portion of a split (or trans) intein (C-intein or I_{C}) and on the other end with a nucleotide sequence encoding the amino-terminal portion of a split intein (N-intein or I_{N}). Expression of the construct within a host system, such as bacteria or eukaryotic cells described herein, results in the production of a fusion protein. The two split intein compounds (i.e., I_{C} and I_{N}) of the fusion protein then assemble to form an active enzyme that splices the amino and carboxy termini together to generate a backbone cyclic peptide. Cyclic polypeptides can be generated using a variety of inteins. Methods of generating cyclic proteins can be found in the art, for example, in WO 00/36093 and WO 01/57183, incorporated herein by reference in their entirety.

As used herein, the term “intein” refers to a naturally-occurring or artificially-constructed polypeptide embedded within a precursor protein that can catalyze a splicing reaction during post-translation processing of the protein.

In one embodiment, an assay is a cell-based assay in which one or more of a normal cell, a precancerous cell, a cancer cell and/or a cell infected with one or more microorganism is contacted with a modulator and the ability of the compound described herein to increase or decrease cell proliferation or the production of target proteins is determined by comparing the levels of binding of the multivalent metal oxide compounds of the present invention to target proteins in a standard sample and a test sample including the modulator. To facilitate detection of the bound multivalent metal oxide compounds, the multivalent metal oxide compounds may be labeled with a radioisotope or enzymatic label such that binding of the compound to the cell can be determined by detecting the labeled compound. For example, compounds can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product. Labeling of compounds of the invention are described further below.

Alternatively, it is also within the scope of this invention to determine the ability of a compound of the invention to interact with a sample without the labeling of any of the interactants. Such assays are described further below.

In yet another embodiment, an assay of the present invention is a cell-free assay comprising contacting a sample with a modulator and determining the ability of the modulator to modulate aberrant cell proliferation and/or infection with one or more microorganisms as described above. Determining the ability of the modulator to modulate aberrant cell proliferation and/or infection with one or more microorganisms can be accomplished by determining the ability of the compounds of the invention to bind to or interact with one or more target proteins the sample in the presence of the modulator.

In certain embodiments of the assay methods of the present invention, it will be desirable to enhance detectability of the compounds described herein (i.e., multivalent metal oxides). In one aspect, the multivalent metal oxide can serve as a nucleating center for formation of grains of reduced metal such as, for example, reduced silver or gold. Silver and gold enhancing products are commercially available. Such enhancement is useful for light microscopy, electron microscopy, Western Blots, detecting cell surface binding, detecting binding in solution and the like. Silver and/or gold enhancement increases the detectability of the multivalent metal oxide by light microscopy. Epipolarized light (e.g., UV light) may be used to increase sensitivity approximately ten-fold. Silver and/or gold enhancement is also useful for producing an intense, sharp, black signal on specimens, proteins immobilized on membranes (e.g., Western blots), cells and the like.

In one embodiment of the present invention, it may be desirable to immobilize the compound (e.g., multivalent metal oxide) to facilitate separation of bound from unbound target proteins, as well as to accommodate automation of the assay. Interaction of the multivalent metal oxide with a target molecule in the presence and absence of a candidate compound, can be accomplished in any vessel suitable for containing the reactants. Examples of such vessels include microtitre plates, test tubes and microfuge tubes. In one embodiment, the multivalent metal oxide can be adsorbed onto beads, such as magnetic beads, or derivatized microtitre plates, which are then combined with the modulator and the sample, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of multivalent metal oxide binding or activity determined using standard techniques.

This invention further pertains to novel agents identified by the above-described screening assays. Accordingly, it is within the scope of this invention to further use an agent identified as described herein in an appropriate animal model. For example, a modulator as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel modulators identified by the above-described screening assays for treatments of disorders associated with aberrant cellular proliferation and/or infection with one or more microorganism as described herein.

Monitoring of Effects During Clinical Trials

Monitoring the influence of modulators (e.g., drugs) on the level of binding of multivalent metal oxides to target proteins in a sample can be applied not only in basic drug screening, but also in clinical trials. For example, the effectiveness of an agent determined by a screening assay as described herein to decrease the level multivalent metal oxide binding can be monitored in clinical trials of subjects exhibiting aberrant cellular proliferation or infection with one or more microorganism.

In one embodiment, the present invention provides a method for monitoring the effectiveness of treatment of a subject with a modulator (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting multivalent metal oxide binding in the preadministration sample; (iii) obtaining one or more post-administration samples from the subject; (iv) detecting the level of multivalent metal oxide binding in the post-administration samples; (v) comparing the level of multivalent metal oxide binding in the pre-administration sample with the multivalent metal oxide binding in the post administration sample or samples; and (vi) altering the administration of the agent to the subject accordingly. For example, increased administration of the agent may be desirable to decrease the multivalent metal oxide binding to lower than detected, i.e., to increase the effectiveness of the agent. Alternatively, decreased administration of the agent may be desirable to increase multivalent metal oxide binding to higher levels than detected, i.e. to decrease the effectiveness of the agent. According to such an embodiment, multivalent metal oxide binding may be used as an indicator of the effectiveness of an agent, even in the absence of an observable phenotypic response.

Pharmaceutical Compositions

Methods of administering one or more of the compounds described herein (e.g., multivalent metal oxides) to an individual include providing pharmaceutically acceptable compositions. In one embodiment, pharmaceutically acceptable compositions comprise a detectable amount of one or more of the compounds described above, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. In one embodiment, the compound is administered orally. In another embodiment, the compound is administered during a surgical procedure (e.g., by syringe, pipette, sponge, aerosol spray, non-aerosol spray and the like). The compounds of the invention can be formulated as compositions for administration to a subject, e.g., a mammal, including a human.

The compounds (e.g., multivalent metal oxides) of the invention are administered to subjects in a biologically compatible form suitable for pharmaceutical administration in vivo. By “biologically compatible form suitable for administration in vivo” is meant a compound to be administered in which any toxic effects are outweighed by the therapeutic effects of the compound. The term “subject” is intended to include living organisms such as mammals. Examples of subjects include, but are not limited to, humans, monkeys, pigs, dogs, cats, rabbits, mice, rats, frogs, toads and transgenic species thereof. Administration of a detectable amount of the compounds of the present invention is defined as an amount effective, at dosages and for periods of time necessary to achieve the desired result. For example, a detectable amount of a compound of the invention may vary according to factors such as the disease state, age, sex, and weight of the individual. Dosage regimes may be adjusted to provide optimum detectability.

The compound may be administered in a convenient manner such as by injection (subcutaneous, intravenous, etc.), oral administration, inhalation, transdermal application, or rectal administration. A compound of the invention can be administered to a subject in an appropriate carrier or diluent, or in an appropriate carrier such as liposomes. The term “pharmaceutically acceptable carrier” as used herein is intended to include diluents such as saline and aqueous buffer solutions. To administer a compound of the invention by other than parenteral administration, it may be necessary to coat the compound with, or co-administer the compound with a material to prevent its inactivation. Liposomes include water-in-oil-in-water emulsions as well as conventional liposomes (Strejan et al. (1984) J. Neiuroimmunol. 7:27). The compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (e.g., antibody) plus any additional desired ingredient from a previously sterile-filtered solution thereof.

In one embodiment, aerosols may be used. Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

When the compound is suitably protected, as described above, the composition may be orally administered, for example, with an inert diluent or an assimilable edible carrier. As used herein “pharmaceutically acceptable carrier” includes any and all solvents, dispersion media, coatings, antibacterial and antifingal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

The following examples are provided for exemplification purposes only and are not intended to limit the scope of the invention which has been described in broad terms above.

### EXAMPLE I

### Diagnostic Testing Using Compounds of the Invention

Diagnostic testing using the compounds described herein may be performed using a one step or a two step process. However, additional steps are not excluded from the practice of the present invention as will be recognized by those of skill in the art. One or more of the steps of the diagnostic tests described herein may be automated using methods known to those in the art.

First Step

The first step, which may optionally be the only step required, is termed the selection stage. As used herein, the phrase “selection stage” is intended to refer to the step wherein the interaction of one or more of the compounds described herein with one or more cells being tested occurs. In one embodiment, the interaction involves one or more compounds described herein binding with targeted chemicals on certain cell surfaces (i.e., the surfaces of selected cells) as described herein. A chemical reaction that occurs with the targeted chemical makes them detectable using analytical equipment and/or additional chemical reagents.

Second Step

The second step is the enhancement stage. As used herein, the phrase “enhancement stage” is intended to refer to the step wherein the interaction of additional chemical reagents that are added to the first step process occurs. The reagents are added to the first step reaction causing the selected cells and/or proteins in step one to be highlighted, i.e., visually observable with the naked eye without the need of analytical equipment. In certain aspects, a combination of analytical equipment and the two step process is used.

One Step Process—General Procedure

A compound of the invention (e.g., a multivalent metal oxide) is added to distilled water to achieve a soluble compound saturation level (SCSL). A fixed amount of the SCSL is added to a prepared sample of tissue (e.g., human tissue) for a specific amount of time. In one aspect, the SCSL is in contact with the tissue sample for a time period between one and ten minutes, depending on the concentration of SCSL. Binding of the SCSL may be analyzed, for example, by analytical equipment.

Two Step Process for Aqueous Testing—General Procedure

A compound of the invention (e.g., a multivalent metal oxide) is added to distilled water to achieve an SCSL. A fixed amount of the SCSL is added to a prepared sample of tissue (e.g., human tissue) for a specific amount of time. In one aspect, the SCSL is in contact with the tissue sample for a time period between one and 45 minutes, depending on the concentration of SCSL. A fixed amount of secondary reagent is added to the SCSL/tissue mixture for an exact timed reaction of between one and fifteen minutes. The reaction of the secondary reagent with the SCSL/tissue mixture may be analyzed, for example, by either analytical equipment or by human visualization.

Two Step Process for Non-aqueous Testing—General Procedure

A compound of the invention (e.g., a multivalent metal oxide) is added to distilled water to achieve an SCSL. A fixed amount of the SCSL is added to a prepared sample of tissue (e.g., human tissue) fixed to a slide (e.g., a glass slide or a deparaffinized slide) for a specific amount of time. In one aspect, the SCSL is in contact with the tissue sample for a time period between one and ten minutes, depending on the concentration of SCSL. The slide is then rinsed off well with water one to three times to assure no residual SCSL left on the sample or the slide. Subsequently, a fixed amount of secondary reagent is added to the slide for an exact timed reaction of between one and 45 minutes. The resulting reaction may then be analyzed, for example, by analytical equipment or by human visualization.

### EXAMPLE II

### Cervical Screening

Spectrophotometric Analyses

Human cervical tissue samples preserved in aqueous solution were placed in a test tube and step one was conducted as described herein. The samples were analyzed with a spectrophotometer. The results were accurate more than 70% of the time for distinguishing among different grades of cells: negative, high grade dysplasia and cancer.

Grown HeLa cell line samples were placed in a test tube with an aqueous solution and step one was conducted as described herein. The samples were analyzed with a spectrophotometer. The results were accurate more than 70% of the time for distinguishing between negative cell lines and cancerous HeLa cell lines.

Human cervical tissue samples preserved in aqueous solution were placed in a test tube and the two step process for aqueous testing described herein was conducted. The samples were analyzed with a spectrophotometer. The results were accurate more than 70% of the time in distinguishing among different grades of cells: non-cancerous (negative control), high grade dysplasia and cancer.

Grown HeLa cell line samples were placed in a test tube with an aqueous solution and the two step process for aqueous testing described herein was conducted. The samples were analyzed with a spectrophotometer. The results were accurate more than 70% of the time for distinguishing between non-cancerous (negative control) cell lines and cancerous HeLa cell lines.

Test Tube Color Analysis Using the Naked Eye

One method involved taking grown cell line samples, placing them in a test tube with an aqueous solution, conducting the two step process for aqueous testing described herein, and viewing the coloration results with the naked eye. The results were accurate more than 90% of the time for distinguishing between negative cell lines and cancerous HeLa cell lines.

Another method involved taking human cervical tissue samples preserved in aqueous solution, placing them in a test tube, conducting the two step process for aqueous testing described herein, and viewing the coloration results with the naked eye. The results were accurate more than 60% of the time for distinguishing among different grades of cells: non-cancerous (negative control), high grade dysplasia and cancer.

Thinprep Slide Analysis Using the Naked Eye

One method involved taking grown cell line samples, fixing them to glass slides, conducting the two step process for non-aqueous testing described herein, and viewing the coloration results with the naked eye. The results were accurate more than 90% of the time for distinguishing between non-cancerous (negative control) cell lines and cancerous HeLa cell lines.

Another method involved taking human cervical tissue samples preserved in aqueous solution, fixing them to glass slides, conducting the two step process for non-aqueous testing described herein, and viewing the coloration results with the naked eye. The results were accurate more than 80% of the time for distinguishing between different grades of cells: non-cancerous (negative control), atypical squamous cells of uncertain significance (ASCUS), low grade dysplasia, high grade dysplasia and cancer. The slides from this test were further processed by either photocopying the actual slides or by computer software enhancement features. The results enhanced the distinction factor.

Thinprep Slide Analysis Using a Microscope

One method involved taking grown cell line samples, fixing them to glass slides, conducting the two step process for non-aqueous testing described above, and viewing the coloration results with a microscope. The results were accurate more than 90% of the time for distinguishing between non-cancerous (negative control) cell lines and cancerous HeLa cell lines.

Another method involved taking human cervical tissue samples preserved in aqueous solution, fixing them to glass slides, conducting the two step process for non-aqueous testing described herein, and viewing the coloration results with a microscope. The results were accurate more than 90% of the time for distinguishing among different grades of cells: non-cancerous (negative control), ASCUS, low grade dysplasia, high grade dysplasia, and cancer.

Paper Analysis Using the Naked Eye

One method involved taking a wooden paddle and adhering an absorbent piece of paper to it, then adhering cancerous HeLa cells to it, and then conducting the two step process for non-aqueous testing described herein. The results were viewed by the naked eye. The darkening of the paper showed presence of cancerous cells, while no discoloration showed non-cancerous (negative control) cells. The results were accurate more than 70% of the time for distinguishing between cancerous and non-cancerous cells.

### EXAMPLE III

### Skin Lesions

Deparaffinized Slide Analysis Using the Naked Eye

One method involved taking deparaffinized skin lesion slides, conducting the two step process for non-aqueous testing described herein, and viewing the results with a microscope. The results were accurate more than 90% of the time for distinguishing between non-malignant nevi lesions and malignant melanoma lesions.

### EXAMPLE IV

### Cervical Exfoliated Cells

This example is directed to a reagent kit to determine the presence of cancer cells in cervical specimens (i.e., Pap (Papanicolaou test) smears). Experiments indicated that tetrasilver tetroxide reagent bound covalently to HeLa cultured cervical cell lines, without binding to normal cells.

Next, HeLa cells were mixed with epithelial (cheek) cells to create a simulated Pap smear. The tests indicated that the tetrasilver tetroxide reagent was able to clearly distinguish the types of cells via selective binding with and darkening of the cytoplasmic membrane of the HeLa cells.

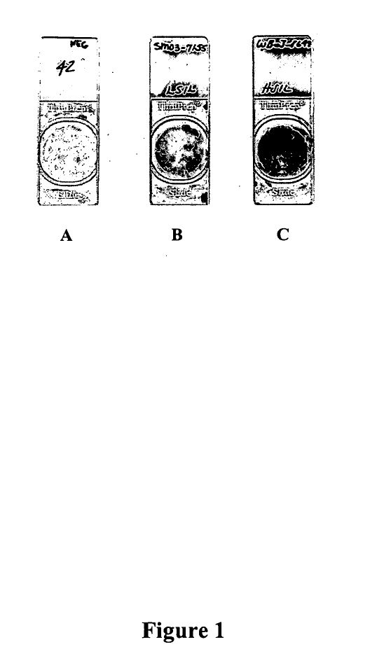

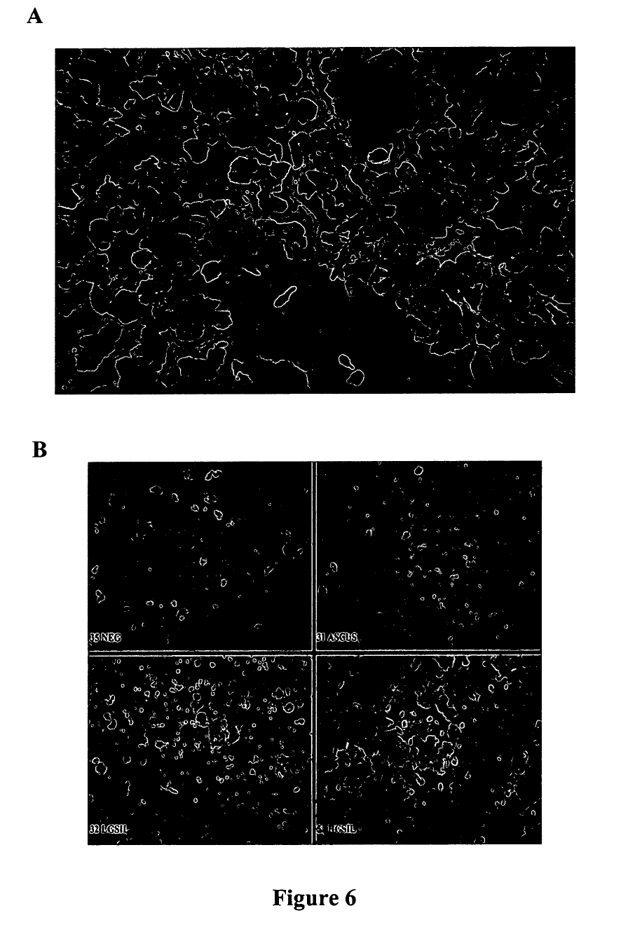

Following these experiments, liquid media containing human cells from Pap smears (thinprep process) were tested with the tetrasilver tetroxide reagent on slides to determine the ability of the reagent to identify the presence of cancerous and potentially precancerous cells (low-grade squamous intraepithelial lesion (LSIL) cells, atypical squamous cells of undetermined significance (ASCUS) and high-grade squamous intraepithelial lesion (HSIL) cells). The test results demonstrated the ability of the tetrasilver tetroxide reagents to modify the color of the thinprep slide media (observable with the naked eye), depending on the degree to which the sample was abnormal. Patient samples that were negative for both cancer and HPV (the human papillomavirus that causes cervical cancer) were visually clear (FIG. 1A). Samples that had been previously classified at LSIL (low grade squamous intraepithelial lesions) were a light brown shade (FIG. 1B). HSIL (high grade squamous intraepithelial) samples were darker (FIG. 1C), and samples bearing cancer cells were nearly black. Similar results are depicted in FIGS. 6A-6B.

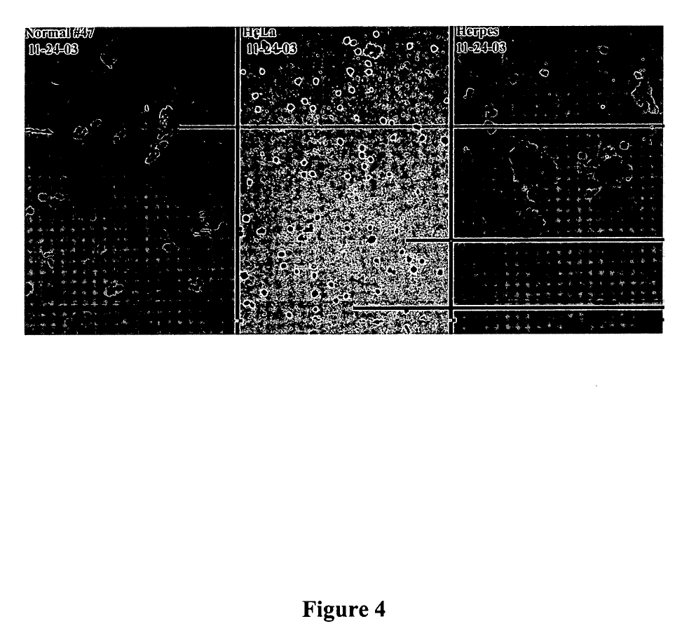

In another experiment, HeLa cells depicted black staining of nuclei, normal (non-cancerous) cells depicted no nuclear staining, and herpes virus infected cells depicted no nuclear staining, but showed a brown staining of the infected cells (FIG. 4).

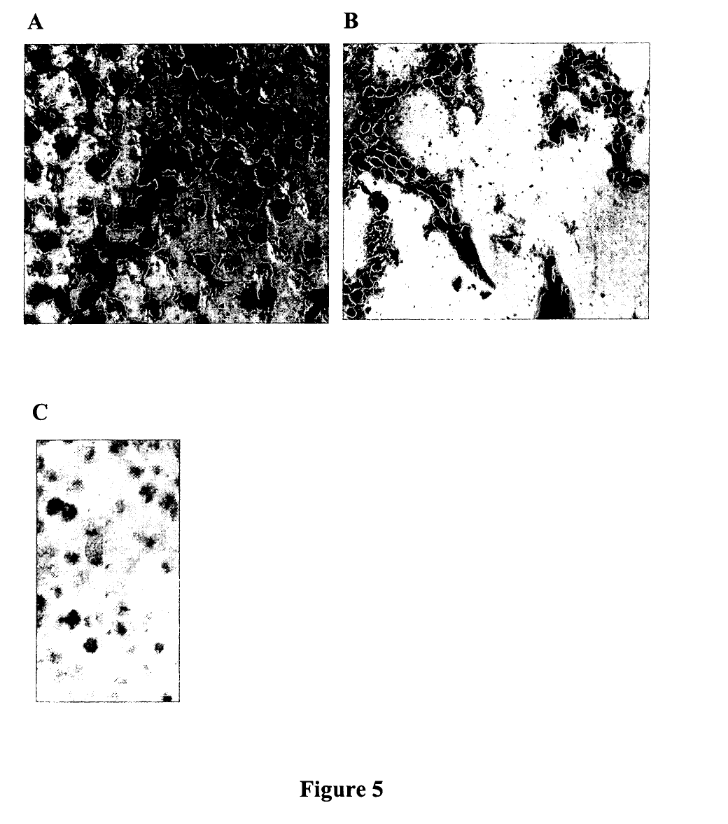

In another experiment, breast cancer cells were similarly selectively stained (FIGS. 5A-5B).

Thus, the tetrasilver tetroxide reagent has the ability to create a color range based on degree of abnormality. Accordingly, aspects of the present invention are directed to a quantitative measure of binding, and therefore detection of degree, of cancerous and precancerous lesions.

### EXAMPLE V

### Skin Biopsy

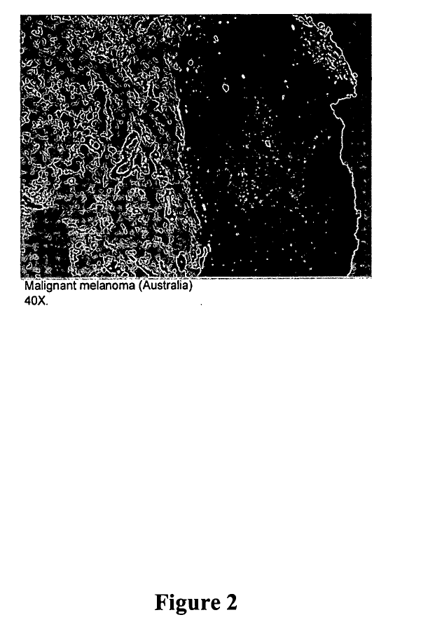

Differentiating between non-malignant nevi and malignant melanoma is a challenge for histologists because of the way that the two mimic each other. Samples were acquired from pathology labs in Germany, Switzerland and Australia and prepared as paraffin sections on slides. Using the sample reagent chemistries as in the cervical application (Example V), a protocol was developed to stain the slide tissue. The results were immediate and definitive. The malignant tissue turned black, while the non-malignant tissue remained within normal parameters (FIG. 2). The control was the diagnosis provided by the histologists using standard techniques. This experiment has been repeated multiple times, each time correctly differentiating the condition of the tissue samples.

### EXAMPLE VI

### Detailed Protocols

I. One Step Process for Spectrophotometer Analysis

Tetrasilver tetroxide (TST) solution (soluble TST) was prepared by adding 5 mg of TST to 1 ml of dd H₂O. Inadvertent “grounding” of the TST was avoided. The solution was mixed gently and thoroughly by pipetting up and down. Formation of air bubbles and excessive agitation were avoided. The solution was centrifuged for 15 seconds at 1,000 rpm to settle excess TST. 500 μl of a human tissue sample was placed in an aqueous solution into a glass tube. 250 μl soluble TST was added to the glass tube (picking up and depositing undissolved TST was avoided). The solution was gently mixed and incubated for five minutes at room temperature. The tube was placed into a spectrophotometer for analysis. Analysis by spectrophotometer was optionally repeated over development time to assure accurate readings.

II. Two Step Process for Spectrophotometer Analysis

TST solution (soluble TST) was prepared by adding 5 mg of TST to 1 ml of dd H₂O. Inadvertent “grounding” of the TST was avoided. The solution was mixed gently and thoroughly by pipetting up and down. Formation of air bubbles and excessive agitation were avoided. The solution was centrifuged for 15 seconds at 1,000 rpm to settle excess TST. 500 μl of a human tissue sample was placed in an aqueous solution into a glass tube. 250 μl soluble TST was added to the glass tube (picking up and depositing undissolved TST was avoided). The solution was gently mixed and incubated for five minutes at room temperature.

The enhancement solution was then prepared. Two drops (approximately 90 μl) each of the 2-part enhancer were added to a microfuge tube. The mixture was vortexed for 40 seconds. 160 μl of enhancement solution was added to the tube and the mixture was incubated at room temperature for 1-2 minutes. The tube was placed into a spectrophotometer for analysis. Analysis by spectrophotometer was optionally repeated over development time to assure accurate readings.

III. Two Step Process for Thinprep Slide Analysis (Naked Eye & Microscope)

TST solution (soluble TST) was prepared by adding 5 mg of TST to 1 ml of dd H₂O. Inadvertent “grounding” of the TST was avoided. The solution was mixed gently and thoroughly by pipetting up and down. Formation of air bubbles and excessive agitation were avoided. The solution was centrifuged for 15 seconds at 1,000 rpm to settle excess TST. Soluble TST was added to a sample slide (picking up and depositing undissolved TST was avoided). The slide was incubated at room temperature for 10 minutes, followed by rinsing by either dipping the slide five times in a beaker of distilled water, or using the dual pipette method to remove excess TST. Dual pipette method: Using two pipettes, water was slowly added to the slide at one corner while the solution was simultaneously drawn up into the other pipette from a distal corner of the sample. Without intending to be bound by theory, the idea was to create somewhat of a flow across the sample, to gently wash it. The sample was quickly and gently blotted once or twice with a cloth wipe.

The enhancement solution was then prepared. One drop each of the 2-part enhancer was added to a microfuge tube. The mixture was vortexed for 40 seconds, and then immediately added it to the sample (an optional very brief centrifuge step at low speed in order to settle the liquid at the bottom of the tube was performed prior to addition to the sample). The sample was incubated at room temperature for 20-30 minutes. Optionally, the progress of the reaction under was observed using a light microscope. The slide was rinsed by either dipping the slide 5 times into a beaker of distilled water, or by the dual pipette method described above. The sample was quickly and gently blotted once or twice with a cloth wipe, and the sample was allowed to dry. The results were observed either under a light microscope or with the naked eye.

For Accelerated Testing Results

TST solution (soluble TST) was prepared by adding 5 mg of TST to 1 ml of dd H₂O. Inadvertent “grounding” of the TST was avoided. The solution was mixed gently and thoroughly by pipetting up and down. Formation of air bubbles and excessive agitation were avoided. The solution was centrifuged for 15 seconds at 1,000 rpm to settle excess TST. Soluble TST was added to a sample slide (picking up and depositing undissolved TST was avoided). The slide was incubated at room temperature for 10 minutes, followed by rinsing by either dipping the slide five times in a beaker of distilled water, or using the dual pipette method to remove excess TST. Dual pipette method: Using two pipettes, water was slowly added to the slide at one corner while the solution was simultaneously drawn up into the other pipette from a distal corner of the sample. Without intending to be bound by theory, the idea was to create somewhat of a flow across the sample, to gently wash it. The sample was quickly and gently blotted once or twice with a cloth wipe.

The enhancement solution was prepared by adding one drop each of the 2-part enhancer to a microfuge tube. The mixture was vortexed for 40 seconds. An ethanolamine dilution was vortexed for 20 seconds, and 20 μl was immediately added to the enhancement solution. This mixture was vortexed for 5 seconds and immediately added to the slide. The slide was incubated at room temperature for 5-10 minutes. Optionally, the progress of the reaction under a light microscope was observed. The sample was quickly and gently blotted once or twice with a cloth wipe and allowed to dry. Results were observed either under a light microscope or with the naked eye.

IV. Two Step Process for Test Tube Color Analysis Using the Naked Eye

TST solution (soluble TST) was prepared by adding 5 mg of TST to 1 ml of dd H₂O. Inadvertent “grounding” of the TST was avoided. The solution was mixed gently and thoroughly by pipetting up and down. The formation of air bubbles and excessive agitation were avoided. The solution was centrifuged for 15 seconds at 1,000 rpm to settle excess TST. 500 μl of a human tissue sample was added to an aqueous solution into a glass tube. 250 μl soluble TST was added to the glass tube (picking up and depositing undissolved TST was avoided). The mixture was gently mixed and incubated for five minutes at room temperature.

An enhancement solution was prepared by placing two drops (approximately 90 μl) each of the 2-part enhancer into a microfuge tube. This mixture was vortexed for 40 seconds. 160 μl of enhancement solution was added to the tube. The tube was incubated at room temperature for 1-15 minutes. Color changes were observed in the tube over time. Pre-cancerous and cancerous cells started out light yellow to light orange, and darkened to brown. Normal cells remained light gray and darkened to green/gray.

V. Process for Paper Analysis Using the Naked Eye

TST solution (soluble TST) was prepared by adding 5 mg of TST to 1 ml of dd H₂O. Inadvertent “grounding” of the TST was avoided. The solution was mixed gently and thoroughly by pipetting the solution up and down. The formation of air bubbles and excessive

TST solution (soluble TST) was prepared by adding 5 mg of TST to 1 ml of dd H₂O. Inadvertent “grounding” of the TST was avoided. The solution was mixed gently and thoroughly by pipetting the solution up and down. The formation of air bubbles and excessive agitation was avoided. The solution was centrifuged for 15 seconds at 1,000 rpm to settle excess TST.

A dip stick was prepared by attaching adhesive enhanced paper to a wooden paddle so that cells would adhere to the paper. Other media may be used to adhere cells including, but not limited to, paper fabric and materials made from natural and/or synthetic fibers. The media could optionally be shaped such that it is suitable for internal use in a subject, e.g., a vaginal insert such as a tampon. The media was removed from a flask or dish containing attached cells (cancerous or non-cancerous). Using the adhesive side of dipstick, firm and complete contact was made with cells without smearing or dragging the dipstick. The dipstick was dried for 5-10 minutes, added to a container containing soluble TST, and incubated for about 5 minutes.

Enhancement solution was prepared by placing four drops (approximately 180 [I) each of the 2-part enhancer into a micro-centrifuge tube. The mixture was vortexed for 40 seconds.

An ethanolamine dilution was vortexed for 20 seconds, and 60 μl was immediately added to the enhancement solution. This mixture was vortexed for 5 seconds. The dipstick was immediately dipped into the ethanolamine/enhancement solution and incubated for 10 minutes. Darkening of the paper was observed over 1 to 10 minutes. Pre-cancerous and cancerous cells lightly darkened the paper to brown or gray. Normal cells remained clear on the paper.

### EXAMPLE VII

### Histological Protocol to Differentiate Malignant from Non-malignant Melanoma

One problem of pathological diagnosis is to consistently distinguish nevi from malignant melanoma. It has been determined that a protocol to identify a target protein at the surface of malignant melanotic neoplasms provides this distinction. In studies performed with a small number of patients, malignant nevi (as well as all malignant melanoma tissue including distant metastases) have reacted to yield an intense dark brown to black coloration whereas non-malignant nevi remain un-reactive. Melanotic normal tissues such as hair shafts did not react. A

Step One: Deparaffinization and Antigen Removal

The following protocol is one method. Other standard methods may also be used. Slides were deparaffinize slowly in three changes of Americlear (Allegiance C4305 ph: 800-964-5227). Slides were slowly hydrated in two changes of absolute alcohol, two changes of 95% reagent alcohol and two changes of distilled water. Slides were placed in Tris buffer for five minutes. Slides were then placed in a glass coplin jar a glass staining dish and treated with hydrogen peroxide for five minutes at room temperature. The slides were then washed well with tap water followed by distilled water.

Working antigen retrieval Citra solution was prepared using the following protocol: 10 ml concentrated Citra (DAKO Target Retrieval Solution Catalog No. S1699 10X conc., ph: 800-235-5763) was added to 90 ml distilled water. Working Citra solution was poured into the appropriate number of coplin jars and the jars were placed in a microwave dish containing 200 ml of tap water. Slides were placed in the Citra and the lids were screwed on the jars (not tightly). The jars were microwaved for 5 minutes (ca. 700 W). The jars were removed from the microwave, and the levels of Citra were brought to the top of the jar. The jars were microwaved again for 5 minutes. The coplin jar(s) were removed from the microwave and from the microwave dish, the lids were removed, and the Citra with slides was allowed to cool at room temperature for 20 minutes. The slides were rinsed well with tap water, followed by distilled water.

Step Two: Treatment with Selection Reagent

The slide(s) were flooded with approximately one mL selection reagent and incubated for ten minutes at room temperature. The slides were washed with five changes of distilled water.

Step Three: Treatment with Enhancer

80 μl of part one enhancer was mixed with 80 μl of part two enhancer, and poured onto slide(s). Color development occurred between 10 and 20 minutes. The slides were washed with water to stop the reaction. Some slides were optionally counterstained with hemadoxalyn. It was not necessary to use cover slip(s), and the reaction product was stable.

Visual Detection

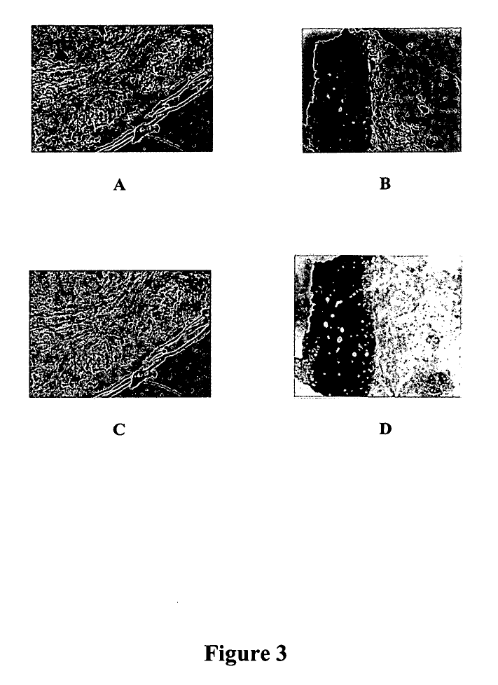

Malignant nevi (as well as all malignant melanoma tissue including distant metastases) yielded an intense dark brown to black coloration whereas non-malignant nevi remained un-reactive (see FIG. 3).

### EXAMPLE VIII

### Tetrasilver Tetroxide and HeLa Cells

Tetrasilver tetroxide was contacted with the human cervical cancer cell line HeLa (available from ATCC(CCL2)). The cells were grown at 37° C. in Eagle's essential medium supplemented with bovine calf serum (GIBCO) and gentamicin sulfate (50 μg/mL, Sigma), pH 7.4. Cell concentration was 100 mg (wet weight)/mL and the cell harvesting buffer used was KCl, NaCl, diNa phosphate and Tris.

NADH oxidase (NOX) activity was determined (as the disappearance of it measured spectrophotometrically at 340 nm) in a reaction mixture (25 mM Tris-MES buffer at pH 7.2, KCN 1 mM) to inhibit low levels of mitochondrial oxidase activity, and 150 micromoles NOX at 37° C. with stirring. Activity was measured with pairs of Hitachi U3210 Spectrophotometers. Assays were initiated by the addition of NOX and tetrasilver tetroxide (Ag₄O₄) for one minute and were repeated on the same sample every 1.5 minutes over a 45-minute period. A mM absorption coefficient of 6.2 was used to determine specific activity.

Protein disulfide-thiol interchange activity associated with tNOX was measured using the dipyridyldithio substrate dithiodipyridine (DTDP). The assay was performed in 50 mM Tris-MES buffer (pH 7.0) and was initiated by the addition of 0.5 micromoles DTDP in 5 microliters DMSO as a control. The reaction was monitored by tracking an increase in absorption at 340 nm using DTDP, specific activity being calculated using a mM absorption coefficient of 6.2. The spectrophotometric assays were performed simultaneously with the two spectrophotometers and DMSO controls. The results indicated that tNOX activity was inhibited completely with tetrasilver tetroxide at a concentration of one part per million (PPM).

The procedure was repeated with plasma protein derived from soybean seeds containing CNOX growth regulator. It was again repeated with a non-cancer cell line MCF-10A of human mammary epithelia having CNOX. The CNOX was not affected at all even at elevated concentrations far above the 1 PPM level. Thus, Tetrasilver Tetroxide was ascertained as a diagnostic reagent which could differentiate between benign and malignant tumors.

### EXAMPLE XI

### Tetrasilver Tetroxide and Mammary Cells

The procedure set forth in Example X was repeated except that the mammary cancer cell line BT-20 was utilized. The results were the same as in Example VIfI.

### EXAMPLE X

### Tetrasilver Tetroxide and Scanning Electron Microscopy

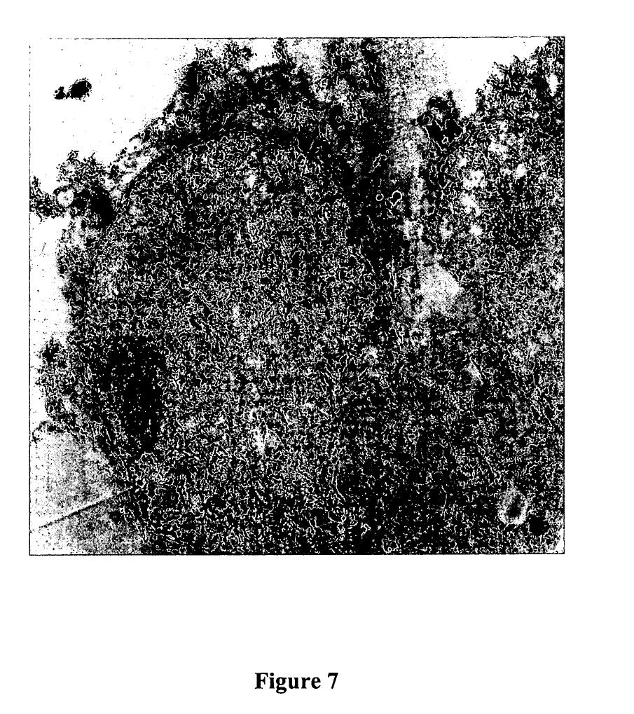

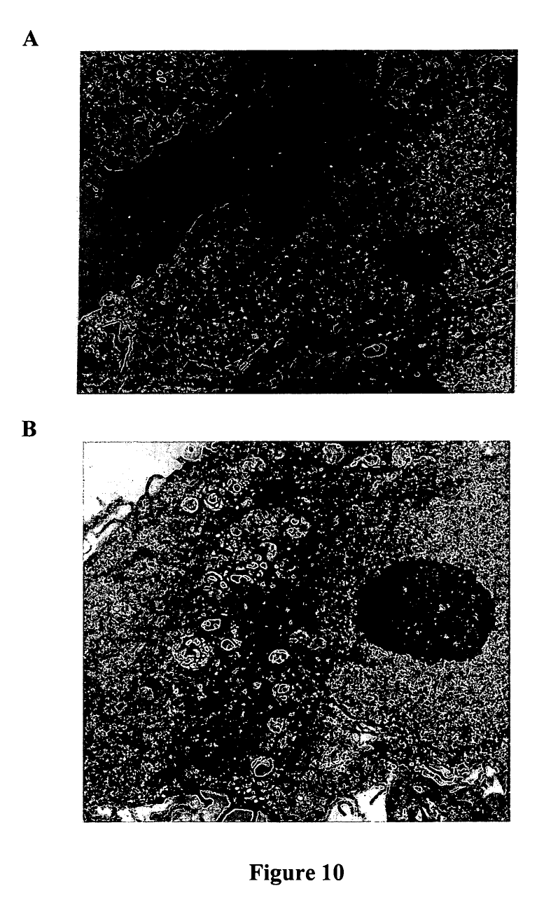

A non-spectrophotometric characterization technique, i.e., scanning electron microscopy (SEM) was used with the procedure set forth in Example VIII. Results obtained by SEM visually indicated the actual binding of tetrasilver tetroxide to tumor cells that was indicative of tNOX inhibition (FIG. 7). Results obtained by SEM also visually indicated the non-binding of tetrasilver tetroxide to non-tumor cells, and hence lack of inhibition, to CNOX (FIGS. 10A-10B). Thus, tetrasilver tetroxide binding may be determined by SEM to differentiate between tumor cells and non-tumor cells.

### EXAMPLE XI

### Enhancement of Tetrasilver Tetroxide

Enhancement can be performed by reducing silver from one solution (i.e., enhancer solution) by another solution (i.e., initiator solution) in the presence of colloidal particles attached to a cell surface. Silver enhancer reagents are available from several sources, including Amersham Biosciences (Piscataway, N.J.), SPI Supplies (West Chester, Pa.), Nanoprobes, Inc. (Yaphank, N.Y.), Kirkegaard and Perry Laboratories, Inc. (KPL) (Gaithersburg, Md.) and Sigma-Aldrich (St. Louis, Mo.). The reaction will cause silver to build up on the surface of the attached particles. Enhancement is rapid but easily controllable within a time span of minutes. Without intending to be bound by theory, amplifications of ten to 100-fold should readily be achieved. Without intending to be bound by theory, the reaction is anticipated to be insensitive to light, can be stopped by washing in water and needs no fixing. The protocol will be applicable to all tetrasilver tetroxide-labeled sections of tissue, whole cells, smears and the like mounted on glass slides, cell monolayers, tissue slices and the like.

Amplification will be detected as an intense brown/black stain at the site of the tetrasilver tetroxide. Amplification can be monitored on the microscope during the reaction. Enhancement protocol will be to mix together one drop of each solution (enhancer and initiator) and applying them to the slide. The reaction may be performed with a cover slip in place to allow high magnification viewing (e.g., by oil immersion). The reaction may be stopped by washing with water. At this point, the reaction may be continued by adding fresh solutions. After washing, slides may be counterstained and mounted using art recognized methods. Without intending to be bound by theory, due to the discrete nature of the growth of the silver, no diffusion of signal should occur. Also without intending to be bound by theory, the silver stain should produce a permanent, non-fading label of sharp resolution and high contrast in bright field viewing.

Other suitable enhancers include, but are not limited to, copper enhancers, selenium enhancers, semiconductor metal enhancers and the like. Additional enhancers would be readily known to one of skill in the art using the disclosure provided herein.

### EXAMPLE XII

### Analysis of Frozen Liver Tissue

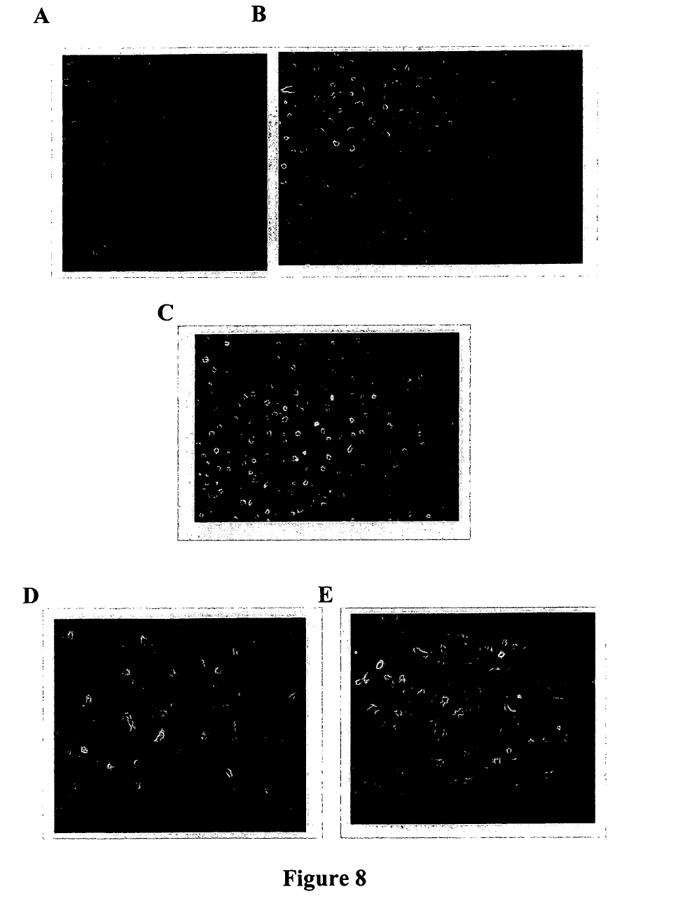

Frozen liver slices were obtained, and the two step process described below for non-aqueous testing was performed. The results were viewed with a microscope and computer enhanced. Frozen liver slices from a wild type mouse (non-cancerous) were compared with frozen liver slices from a transgenic mouse expressing tNOX on its liver cells. Using a microscope and computer photography enhancement, the transgenic liver showed black nuclei and black borders whereas the wild type mouse liver did not (FIGS. 8A-8E).

Two Step Process for Frozen Tissue Sections Analysis

TST solution was prepared by adding 5 mg of TST to 1 ml of dd H₂O. Inadvertent “grounding” of the TST was avoided. The solution was mixed gently and thoroughly by pipetting up and down. Air bubbles and excessive agitation were avoided. The solution was centrifuged for 15 seconds at 1,000 rpm to settle excess TST.

Soluble TST was added to a sample section, while avoiding undissolved TST. The sample was incubated at room temperature for 10 minutes. Excess TST was removed by dipping the section several times in a beaker of distilled water. The sample was quickly and gently blotted once or twice with a cloth wipe.

The enhancement solution was prepared by placing one drop each of the two-part enhancer into a microfuge tube. The mixture was vortexed for 40 seconds, and immediately added to the sample. The solution was optionally centrifuged briefly at low speed in order to settle the liquid at the bottom of the tube prior to addition to the sample. The sample was incubated at room temperature for 20-30 minutes. The sample was rinsed by dipping the slide several times into a beaker of distilled water. The sample was quickly and gently blotted once or twice with a cloth wipe and allowed to dry. Results were observed under a microscope.

### EXAMPLE XIII

### Bacterial Analyses

Analyses of Bacteria in an Aqueous Solution:

Ampicillin resistant bacteria were placed in a phosphorus buffer solution in a test tube, and the two step process for aqueous testing described below was conducted. Coloration results were determined with the naked eye. The results indicated that the control (phosphorous buffer and two step process) was clear to cloudy/white, but the ampicillin resistant bacteria were translucent pink.

Two Step Process for Test Tube Color Analysis Using the Naked Eye

TST solution (soluble TST) was prepared by adding 5 mg of TST to 1 ml of dd H₂O. Inadvertent “grounding” of the TST was avoided. The solution was mixed gently and thoroughly by pipetting up and down. Air bubbles and excessive agitation were avoided. The solution was centrifuged for fifteen seconds at 1,000 rpm to settle excess TST. 500 μl of an ampicillin resistant bacteria sample was added to a phosphorus buffer solution in a glass tube. 250 μl soluble TST was added to the glass tube, while avoiding the addition of undissolved TST. The solution was gently mixed and incubated for five minutes at room temperature.

The enhancement solution was prepared by placing two drops (approximately 90 [I) each of the two-part enhancer solutions into a microfuge tube. The mixture was vortexed for 40 seconds. 160 μl of enhancement solution was added to the tube and the mixture was incubated at room temperature between five and fifteen minutes. Color changes were observed in the tube over time. If bacterial cells were present, the liquid turned translucent pink. If bacterial cells were not present, the liquid remained clear to cloudy/white.

### EXAMPLE XIV

### Fe₃O₄Staining of HeLa Cells

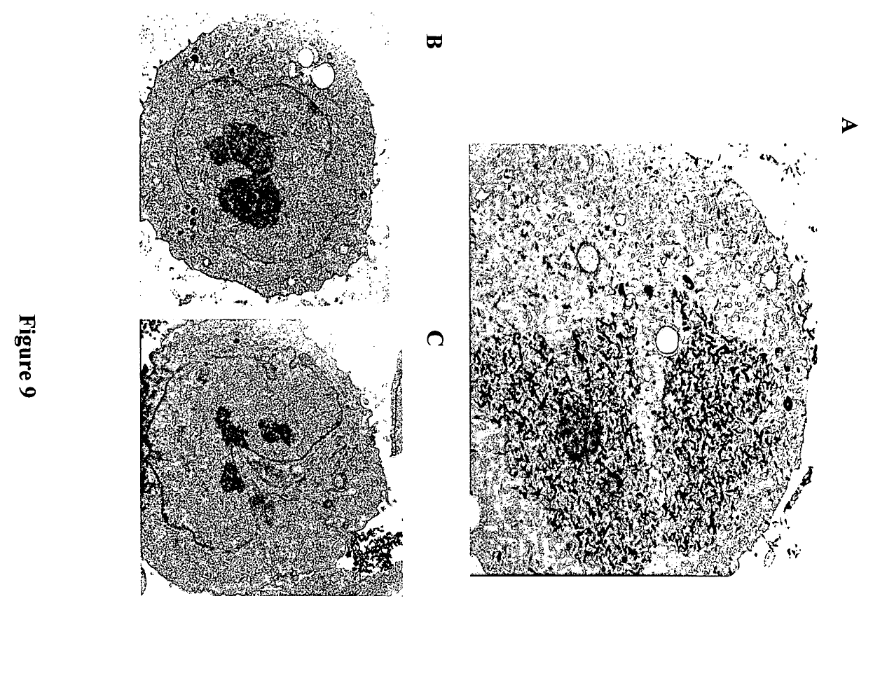

HeLa cells were selectively stained with Fe₃O₄ using the protocols set forth herein. FIGS. 9A-9C depict the results of such an experiment.

### Equivalents

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference herein in their entirety for all purposes to the same extent as if each individual publication or patent application were specifically indicated to be so incorporated by reference.

What is claimed is:

1. A method for detecting abnormal cells comprising:
obtaining a sample;
contacting the sample with a multivalent metal oxide; and
detecting binding of the multivalent metal oxide to an abnormal cell or a protein associated with an abnormal cell.

2. The method of claim 1, wherein the sample is selected from the group consisting of muscle tissue, cervical tissue, skin tissue, spinal tissue and liver tissue.

3. The method of claim 1, wherein the sample is selected from the group consisting of blood cells, fat cells, cervical cells, cheek cells, throat cells, mammary cells, muscle cells, skin cells, liver cells, spinal cells and bone marrow cells.

4. The method of claim 1, wherein the multivalent metal oxide is selected from the group consisting of Ag₄O₄, Bi₂O₄, CO₃O₄, Fe₃O₄, Mn₃O₄, Pr₆O₁₁ and Tb₄O₇.

5. The method of claim 4, wherein the multivalent metal oxide is Ag₄O₇.

6. The method of claim 1 wherein the protein is tNOX.

7. A method for detecting a microorganism in a sample comprising:
obtaining a sample;
contacting the sample with a multivalent metal oxide; and
detecting binding of the multivalent metal oxide to a microorganism, wherein binding occurs when a microorganism is present in the sample.

8. The method of claim 7, wherein the sample is a bodily fluid.

9. The method of claim 8, wherein the bodily fluid is selected from the group consisting of sputum, lymph, blood, urine, tears, breast milk, nipple aspirate fluid, seminal fluid, vaginal secretions, feces, cerebrospinal fluid, peritoneal fluid, pleural fluid, pus and ascites.

10. The method of claim 7, wherein the multivalent metal oxide is selected from the group consisting of Ag₄O₄, Bi₂O₄, CO₃O₄, Fe₃O₄, Mn₃O₄, Pr₆O₁₁ and Tb₄O₇.

11. The method of claim 10, wherein the multivalent metal oxide is Ag₄O₄.

12. The method of claim 7, wherein the microorganism is selected from the group consisting of viruses, bacteria, fungi and parasites.

13. A method for diagnosing a disorder associated with aberrant cellular proliferation in a subject comprising:
obtaining a sample from the subject;
contacting the sample with a multivalent metal oxide;
detecting binding of the multivalent metal oxide to the sample; and
diagnosing a disorder associated with aberrant cellular proliferation if binding occurs.

14. The method of claim 13, wherein the disorder is cancer.

15. The method of claim 13, wherein the sample is selected from the group consisting of muscle tissue, cervical tissue, skin tissue, spinal tissue, liver tissue.

16. The method of claim 13, wherein the sample is selected from the group consisting of blood cells, fat cells, cervical cells, cheek cells, throat cells, mammary cells, muscle cells, skin cells, liver cells, spinal cells and bone marrow cells.

17. The method of claim 13, wherein the multivalent metal oxide is selected from the group consisting of Ag₄O₄, Bi₂O₄, CO₃O₄, Fe₃O₄, Mn₃O₄, Pr₆O₁₁ and Tb₄O₇.

18. The method of claim 13, wherein the multivalent metal oxide is Ag₄O₄.

19. The method of claim 17, wherein the subject is a mammal.

20. The method of claim 19, wherein the mammal is a human.

21. A method for diagnosing a disorder associated with infection by a microorganism in a subject comprising:
obtaining a sample from the subject;
contacting the sample with a multivalent metal oxide;
detecting binding of the multivalent metal oxide to the sample; and
diagnosing a disorder associated with infection by a microorganism if binding occurs.

22. The method of claim 21, wherein the disorder is caused by a microorganism selected from the group consisting of viruses, bacteria, fungi and parasites.

23. The method of claim 21, wherein the sample is a bodily fluid.

24. The method of claim 23, wherein the bodily fluid is selected from the group consisting of sputum, lymph, blood, urine, tears, breast milk, nipple aspirate fluid, seminal fluid, vaginal secretions, feces, cerebrospinal fluid, peritoneal fluid, pleural fluid, pus and ascites.

25. The method of claim 21, wherein the multivalent metal oxide is selected from the group consisting of Ag₄O₄, Bi₂O₄, CO₃O₄, Fe₃O₄, Mn₃O₄, Pr₆O₁₁ and Tb₄O₇.

26. The method of claim 24, wherein the multivalent metal oxide is Ag₄O₄.

27. The method of claim 21, wherein the subject is a mammal human.

28. The method of claim 27, wherein the mammal is a human.

29. A kit for detecting or monitoring aberrant cellular proliferation comprising a multivalent metal oxide and instructions for use.

30. A kit for detecting or monitoring a microorganism comprising a multivalent metal oxide and instructions for use.

31. A kit for diagnosing or monitoring a disorder associated with aberrant cellular proliferation comprising a multivalent metal oxide and instructions for use.

32. A kit for diagnosing or monitoring a disorder associated with infection by a microorganism comprising a multivalent metal oxide and instructions for use.
